(19) United States
(12) Patent Application Publication
(10) Pub. No.: US 2016/0353786 A1
(43) Pub. Date: Dec. 8, 2016

(54) Compositions and Methods to create natural curing foods

(71) Applicant: Elizabeth McHugh, Guttenberg, NJ (US)

(72) Inventor: Elizabeth McHugh, Guttenberg, NJ (US)

(21) Appl. No.: 15/130,956

(22) Filed: Apr. 16, 2016

Related U.S. Application Data
(60) Provisional application No. 62/148,693, filed on Apr. 16, 2015.

(51) Int. Cl. A23L 7/113; A23L 7/117; A61K 36/899; A23L 31/00; A23L 33/00; A21D 13/00; A23L 17/60

(52) U.S. Cl. CPC A23L 7/113; A21D 13/007; A21D 13/0074; A21D 13/0019; A23L 7/117; A23L 17/60; A23L 31/00; A23L 33/00; A61K 36/899; A23V 2002/00

(57) ABSTRACT

A natural food composition or nutraceutical compositions comprising a compound or a mixture of compounds selected from the group consisting of organic super foods, without any genetically modified or toxic ingredients, all prepared mechanically, thereby, these nutraceutical compositions act as medicine, to control symptoms, prevent, and treat a broad range of health issues. In addition to providing daily nutritional value, needed in the human body, to promote general well-being, my invention is distinguished from prior art, in the way, my methods of integrative approaches use manufactured thought-out mechanical processes, without high temperatures, that protect food from losing their nutrients and medicinal properties, whereby, becoming “disease preventive food” as “dietary and culinary interventions” with “real food or mechanically processed food” designed as a safe and cost-effective alternative to drugs. This is a brilliant solution for prevention and intervention -focused food against chronic diseases in the world.

### TECHNICAL FIELD OF THE INVENTION

This invention generally relates to the field of nutrition, specifically nutraceutical compositions made from organic super foods, designed as “disease preventive food” for dietary and culinary intervention”. These shall contain all the nutrients needed to provide a daily nutritious, balanced diet in humans that promote general well-being. Such foods can be manufactured with purposely planned cooking methods and mechanical processes, without genetic modification or toxic ingredients that protect the food from losing their nutrients and medicinal properties.

### BACKGROUND OF THE INVENTION

Literature surveys on similar foods that are also used as medicines, are found to be “chemically processed food” which embodies various inherent negativities. These being
Chemically processed food is killing humanity considering the fact that a human's body is a complex machine.
“There is no food-based strategy for disease prevention”
“Growth of chronic diseases burdens the world”.
   Also, no other invention planned to create products or formulas as “disease preventive food” for “dietary and culinary interventions”, that contain the total nutrients needed to provide a daily balanced diet, or manufactured with thought-out mechanical processes that protect the food from genetic modification or toxicity from losing their nutrients and medicinal properties, thus helping to fix challenge, as explained below:

Challenges:

Challenge #1: The human body is a complex machine consisting of many separate, interconnected, individually vibrating machines. It is a chemical and mechanical factory within. It is most difficult to maintain so many inter-related processes and parameters, even with the most advanced computing power. The entire world has taken this challenge very lightly, without giving importance to the human need for the critical knowledge, to make people aware of the consequences of poor daily nutrition and how deficiencies slowly make us sick.

Challenge #2: “Chemically processed food is killing humanity”. With nearly 7 out of 10 Americans being overweight, 1 in 4 being affected with diabetes or pre-diabetes, 2 of every 3 people is diagnosed with cancer today, the heavily processed standard American diet, SAD, is clearly a devastating factor in today's and the future generation's health.

2.1 Lots of Calories for Little Nutrition: Highly Processed foods over-stimulate the production of dopamine, aka the “pleasure” neurotransmitter, which can cause excessive cravings, leading to obesity, diabetes and cancer. A fast-food burger plus fries and coke count to almost an entire days worth of required calories, causing excess weight gain without receiving the vitamins and minerals, the live enzymes and micronutrients, the high-quality protein or healthy fats the body needs to function and thrive.

2.2 Dangerous Food Additives: Many food additives—preservatives, flavorings, colors and other ingredients—while legal, they are not safe for long-term consumption. Some, such as sodium nitrate, BHA, BHT, aspartame, Blue 1, 2, and potassium bromate, have been linked to an increased risk of cancer. Studies show that various common food dyes, and the preservative, sodium benzoate—found in soft drinks, fruit juices and salad dressings—cause some children to become more hyperactive and distractible. Phosphate additives magnify taste, texture, and shelf-life, but its effects cause cardio-vascular damage, rapid aging, kidney failure, and weak bones.

2.3 Fructose: Fructose, now the number one calorie source in the US, diminishes feelings of fullness since it does not stimulate a rise in leptin, a hunger and fat storage regulator in the body. Fructose also reduces the amount of leptin crossing the blood-brain barrier by raising triglycerides. Leptin plays a significant role in the development of heart disease, obesity, diabetes, osteoporosis, autoimmune diseases, reproductive disorders, and rate of aging.

GMO-Genetically Modified (GM) Organisms: Some 75 percent of processed foods contain GM ingredients, which are increasingly linked to serious health problems. The foundation of most processed foods in grocery stores today spring from laboratories, not nature. Genetically-modified organisms (GMOs), have been linked to infertility, organ damage, and cancer.

MSG: Monosodium glutamate (MSG), a flavor enhancer, is added to thousands of processed (canned soups, crackers, meats, salad dressings, frozen dinners, chips etc.), and restaurant foods. MSG is popular, because it enhances the flavor of food, making processed meat, frozen dinners and salad dressings taste fresher, and canned foods less tinny. However, it is also an excitotoxin, which overexcites brain cells causing varying degrees of nerve damage or cell death, and triggers or worsens learning disabilities, Alzheimer's, Parkinson's, Lou Gehrig's diseases and more.

2.9 Fake food spoil your mind: Consuming junk food can cause people to become angry and depressed, have brain fog and difficulty in concentrating. Nutritionally-dense foods levels mood, maintain energy, and fosters productivity.

2.10 Refined food ruin your insides: Alterations and deletions in refined food with their natural fibers, enzymes, vitamins, result in their longer shelf life. Consumption of packaged, processed foods wreaks havoc on the digestive system, pollutes the internal ecosystem, disrupts beneficial bacteria and increases infection.

2.11. Processed foods are filled with pesticides: The GMOs used in processed food are grown by applying glyphosphate along with other pesticides and herbicides, which end up in the crop. Some breakfast cereals out there contain up to 70 different types of pesticides, fumigation chemicals, and other residues.

Challenge #3: There is no food-based strategy for disease prevention. The world culture doesn't have an integrated, chronic disease management model based on primary health care. With food-based strategy of “disease prevention and intervention”, the adverse trends and risk factors in the major chronic diseases can be slowed and reversed, premature deaths and unnecessary disability avoided.

Challenge #4: Unplanned high temperatures lose their nutrients and medicinal properties: People have been accustomed to cooking without awareness of high temperatures destroying the nutrients, and never considered the fundamentals of eating nutritiously. Despite the science of steam cooking being around for centuries and now a growing trend, changing to the ideal steam cooking method to prevent the loss of nutrients, while cooking food is a challenge, therefore, “culinary intervention” is the necessary purpose of the invention, and another “disease prevention strategy”. This is crucial to reverse the worldwide growing crisis of chronic diseases.

Challenge #5: “Growth of chronic diseases burden the world”. Chronic diseases are increasing in global prevalence and have become the dominant health burden in many developing countries and seriously threaten their ability to improve the health of their populations. This rise of lifestyle-related chronic disease in poor countries is the result of a complex constellation of social, economic, and behavioral factors.

The prime ingredient of a healthy lifestyle is to have a balanced diet or healthy eating plan. A healthy diet provides the body with essential nutrition to support energy needs without exposure to toxicity. The basic nutrition that a balanced daily diet must provide, are protein, folic acid, iron, calcium, magnesium, vitamins, enzymes, fibers, essential fatty acids and iodine. To take care of these, it must include several food groups since one single group cannot provide the best nutritious diet required for good health.

### OBJECTIVES OF THE INVENTION

A primary objective of the present invention is to provide nutraceutical formulas or food compositions for improving health, preventing chronic diseases, postponing the aging process, increasing the life expectancy and supporting functions and integrity of the body.

Another objective of the present invention is to provide an alternative solution to chemically processed food wherein, the processing of food is done mechanically to retain the nutritional value of the ingredients used in the processed food.

Another objective of the present invention is to provide a food based strategy for disease prevention.

Still another objective of the present invention is to provide natural food compositions made from super foods, which includes the nutrients needed to provide a daily balanced diet with critical components like proteins, enzymes, minerals, vitamins, fiber, fatty acids and iodine to bring and maintain general well-being of the body.

Another objective of the present invention is to provide“mechanically processed food” or “real food” choices without genetic modification or presence of toxic ingredients like MSG, artificial flavors or sweeteners, preservatives, highly processed trans or hydrogenated fats.

Still further objective of the present invention is to provide a mechanical process of steam cooking which protects the food from losing its nutrients and medicinal properties creating a “culinary intervention” which is the necessary purpose of the invention, and another “disease prevention strategy”.

Another objective of the present invention is to provide mechanically processed food and food compositions in a way that creates “disease preventive food”, as an alternative to drugs.

Another objective of the present invention is to provide for food compositions or nutraceutical compositions that have been designed to meet the daily needs of the complex human body aiding in antioxidant defenses, cell proliferation, gene expression, safeguarding of mitochondrial integrity.

Still further objective of the present invention is to provide an alternative option to processed food.

### SUMMARY OF THE INVENTION

A natural food composition or nutraceutical compositions made from organic super foods comprising of barley, brown rice, oats, quinoa, whole wheat, soybeans, black beans, honey, flaxseed, sunflower seeds, walnuts, black seed, moringa, goji berries, coconut, brown seaweeds and chaga mushrooms. The ingredients are combined in various proportions and in different combinations and prepared as various forms of food products designed as food intervention for disease prevention-focused food that includes the nutrients needed for a daily nutritious balanced diet with critical components like proteins, enzymes, minerals, vitamins, fiber, fatty acids and iodine to bring and maintain general well-being of the human body. These food compositions or nutraceutical compositions without genetic modification or toxic ingredients, are manufactured with thought-out mechanical processes that protect the food from losing their nutrients and medicinal properties.

### DETAILED DESCRIPTION OF THE PREFERRED EMBODIMENTS

In utilization of the above mentioned organic super foods for various food compositions, broken down by element and weight percentage thereof, to form the desired food product are discussed hereof.

These organic super foods or nutraceutical compositions have been purposely designed to improve health, prevent chronic diseases, postpone the aging process, increase life expectancy and support functions and integrity of the body. In time, this nutraceutical intervention will control symptoms, treat, prevent and/or reverse different health issues.

The various effects of the nutritional food included in the present invention have been grouped into separate health groups as under:
HG #1: Inflammatory (Inflammation, joints, neuropathic pain, respiratory)
HG #2: Brain health (General well-being, anxiety, depression, memory, insomnia, stress, alzheimer's and dementia)
HG #3: Circulation (Heart and circulatory health, high blood pressure, cholesterol and triglycerides)
HG #4: Weight loss and stomach health
HG #5: Metabolic (Cancer protection, diabetes, blood sugar control)
HG #6: Healthy skin, nails and hair, teeth and bones
HG #7: Anti-aging, Eye health
HG #8: Hormone balance, Menopause.
HG #9: Immune System (Infections, Colds and Flu)

The following are various food compositions, broken down by element and weight percentage thereof, to form the desired food product and their effects on human health based on their nutritional functions and grouped as above.

### Food Product—For Pasta and Ready to Serve Food

### Formula #-1

### Effecting health group: HG #1, HG #3,HG #4, HG #6, HG #8

| | Percentage by |
|---|---|
| Ingredient | weight (gram) |
| Seaweeds | 10 |
| Black seeds | 24 |
| Sunflower | 100 |
| Flaxseeds | 50 |
| Soyabean | 170 |
| Oats | 120 |
| Barley | 120 |
| Brown Rice | 400 |
| Total | 1000 |

In order to produce a food composition as herein described, the preparation of dough for pasta involves pulverizing the above ingredients and placing & mixing them in a conventional mixing device thereby, slowly adding 400gram water and adding 50 gram of crushed flaxseeds. After waiting for 5 minutes, the next step involves placing the well-blended mixture in the pasta machine for 10 minutes after which the pasta sheets of different lengths and thicknesses gets prepared, whereby the prepared pasta sheets are placed in different machines accordingly, to make the pastas of the desired forms like ravioli, cannelloni, lasagna, long and short pastas. The pasta is then dried for 12 hours. These pastas have no preservatives, therefore it will be packed and sold with expiration date of 12 months. The customer is encouraged to prepare it at home using the steam technique, as used for their fish/vegetables, or at low boiling point/simmer water for 5-6 minutes. Ready to serve food has the same pasta formulas, and is served with raw foods, such as an avocado, papaya or other fresh food containing enzymes.

### Formula #-2

### Effecting Health Group; HG #1, HG #2,HG #3, HG #4, HG #5, HG #6, HG #7

| | Percentage by |
|---|---|
| Ingredient | weight (gram) |
| Chaga Mushroom | 50 |
| Seaweeds | 10 |
| Black seeds | 35 |
| Flax seeds | 100 |
| Moringa | 100 |
| Sunflower seeds | 100 |
| Brown rice | 600 |
| Walnuts | 5 |
| Total | 1000 |

The mixture in this example can be prepared in the same manner as that used for formula 1.

### Formula #-3

### Effecting Health Group: HG #9, HG #1, HG #3, HG #4, HG #5

| | Percentage by |
|---|---|
| Ingredient | weight (gram) |
| Seaweed | 5 |
| Black seeds | 10 |
| Flaxseeds | 100 |
| Echinacea | 15 |
| Oats | 150 |
| Barley | 150 |
| Quinoa | 150 |
| Brown rice | 420 |
| Total | 1000 |

The mixture in this example can be prepared in the same manner as that used for formula 1.

### Formula #-4

### Effecting Health Group: HG #1, HG #2, HG #3, HG #4, HG #5, HG #6, HG #7

| | Percentage by |
|---|---|
| Ingredient | weight (gram) |
| Brown rice | 520 |
| Black beans | 300 |
| flaxseeds | 100 |
| chaga | 50 |
| Black seeds | 25 |
| Seaweeds | 5 |
| Total | 1000 |

The mixture in this example can be prepared in the same manner as that used for formula 1.

### Food Product for Pizza, Bread, Tacos, Tortillas, Crackers, Arepas And Empanadas

### Formula #-1

### Effecting Health Group: HG #1, HG #3, HG #4, HG #6, HG #8

| | Percentage by |
|---|---|
| Ingredient | weight (gram) |
| Seaweeds | 2 |
| Blackseeds | 3 |
| Sunflower Oil | 15 |
| Flaxseeds | 10 |
| Soybean | 20 |
| Brown Rice | 100 |
| Total | 152 |

In order to produce a food composition as herein described, the preparation of dough for bread/pizza/tacos/tortillas/crackers/arepas/empanadas involves pulverizing the above ingredients and placing & mixing them in a conventional mixing device, thereby, slowly adding 90 gram water and adding 20 gram of sunflower oil. The next step involves placing the well-blended mixture in the mixing device for 10 minutes, which produces the required dough for making different pasta shapes for the different products, wherein filling those different products shapes with nutritionally balanced food fillings, thereby producing the final nutraceutical composition for the bread/pizza/tacos/tortillas/crackers/arepas/empanadas.

The dough for crackers, is steam baked for 1-2 minutes and sold as such, whereas, the dough for bread/pizza/tacos/tortillas/arepas/empanadas, thus produced, are frozen and sold, and when ready to be consumed, they are steam baked for 15 minutes at a temperature of 200 degrees Fahrenheit for pizza and bread and 1-2 minutes for tacos, tortillas, arepas and empanadas.

### Formula #-2

### Effecting Health Group: HG #1, HG #2,HG #3, HG #4, HG #5, HG #6, HG #7

| | Percentage by |
|---|---|
| Ingredient | weight (gram) |
| Chaga Mushrooms | 8 |
| Seaweeds | 2 |
| Black seed | 5 |
| flaxseeds | 15 |
| Moringa | 25 |
| Sunflower oil | 13 |
| Walnuts | 2 |
| Barley | 80 |
| Total | 150 |

The mixture in this example can be prepared in the same manner as that used for formula 1.

### Formula #-3

### Effecting Health Group: HG #9, HG #1,HG #3, HG #4, HG #5

| Ingredient | Percentage by weight (gram) |
|---|---|
| Black seeds | 5 |
| Flaxseeds | 5 |
| Echinacea | 10 |
| Oats | 10 |
| Barley | 80 |
| Quinoa | 30 |
| Sunflower oil | 10 |
| Total | 150 |

The mixture in this example can be prepared in the same manner as that used for formula 1.

### Food Product—Cold/Hot Cereals/Snack Bars

### Formula #-1

### Effecting Health Group: HG #1, HG #3,HG #4, HG #6, HG #8

| Ingredient | Percentage by weight (gram) |
|---|---|
| Oats | 600 |
| Barley | 80 |
| Soybean | 80 |
| Brown rice | 50 |
| Flaxseeds | 50 |
| Honey | 50 |
| Sunflower Oil | 50 |
| Blackseeds | 20 |
| Seaweeds | 10 |
| Gogi berries | 10 |
| Total | 1000 |

In order to produce a food composition as herein described, the preparation of the granola for cold cereals and snack bar involves pulverizing the above ingredients, mixing and placing the mixture in a tray on the conveyer belt for wet steam baking at 150 degree Fahrenheit for 1 minute, thence, adding 50 gram of honey and dry steam baking for another minute.

Whereas, for hot cereal, all the above ingredients are combined in a large container and stored in a cool dry condition. Before consumption, water is added to the mixture and steamed in an oven for 3 to 5 minutes. Then, served hot with milk or yogurt, fruits, nuts or seeds.

### Formula #-2

### Effecting Health Group: HG #1, HG #2,HG #3, HG #4, HG #5, HG #6, HG #7

| Ingredient | Percentage by weight (gram) |
|---|---|
| Oats | 600 |
| barley | 90 |
| Brown Rice | 50 |
| Moringa | 50 |
| Chaga Mushrooms | 50 |
| Flax seeds (crushed) | 50 |
| Sunflower Seeds | 50 |
| Gogi Berries | 35 |
| Sea Weeds | 10 |
| Walnuts | 10 |
| Black seeds | 5 |
| Total | 1000 |

The mixture in this example can be prepared in the same manner as that used for formula 1.

### Formula #3

### Effecting Health Group: HG #9, HG #1,HG #3, HG #4, HG #5

| Ingredient | Percentage by weight (gram) |
|---|---|
| Oats | 600 |
| Quiona | 100 |
| Barley | 90 |
| Brown Rice | 80 |
| Honey | 50 |
| Flaxseeds (crushed) | 50 |
| Black seeds | 15 |
| Seaweeds | 5 |
| Coconut | 10 |
| Total | 1000 |

The mixture in this example can be prepared in the same manner as that used for formula 1.

The above nutraceutical compositions are representative of unique formulas designed as “disease preventive food”. In time, this nutraceutical intervention will control symptoms, treat, prevent and/or reverse different health issues.

As stated, various combinations are possible, of the components herein described, in order to provide a food composition, which is capable of serving as a substitute for nutritionally imbalanced, chemically processed food, as an alternative to drugs.

The food compositions, of the instant invention, can be used to provide balanced proper nutrition for all age groups (developing and sustaining) and can be readily adapted to meet any of several nutritional lifestyles, as well as, provide the following benefits to the human body. The separate beneficial properties of the various natural ingredients included in the various food compositions are enumerated below.

SEAWEEDS: Seaweeds have unique mineral content like calcium, copper, and concentrated form of iodine (for healthy metabolism), bioavailable iron, magnesium, manganese, molybdenum, phosphorus, potassium, selenium, vanadium, as found in human blood. Seaweeds also offer a variety of unique phytonutrients, including sulfated polysaccharides, antioxidants (anti-inflammatory), polyphenol antioxidants (flavonoids) along with substantial amounts of protein, niacin, vitamin B1, vitamin B2, vitamin B5, vitamin B6 and B12 and a variety of enzymes (that reduce blood sugar level). Moreover, on account of the sulfated polysaccharides compounds, seaweeds/sea vegetables possess unique anti-inflammatory (especially osteoarthritis), anti-cancer (especially colon and breast cancers), anticoagulant, antithrombotic, and antiviral properties (herpes simplex virus). Seaweeds also acts as a “blood purifier” as well as neutralizing the over-acidic effect of diet by alkalizing blood. With their powerful chelating and detoxifying properties, seaweeds provide protection to environmental toxins and drawing out of body wastes.

MORINGA: Another natural sources of basic nutrients is Moringa Oleifera It contains more than 92 nutrients, 46 types of antioxidants and vitamins A-Z, 36 anti-In ammatones, 18 amino acids and 9 essential amino acids. It cures and prevent over 300 illness including diabetes, high blood pressure, arthritis, stroke and cancers. It is also commonly used for energy, beautiful skin, sleep and strengthening teeth. Besides, stimulating the body to feel good, fresh and energized, moringa has several thousand times more zeatin than any other known plant. It allows new skin cells to grow at a faster rate when old skin cells die. This results in a marked reduction of wrinkles on the face and other parts of the body, and a more youthful skin appearance. Moringa have our times more chlorophyll than Wheatgrass. Sulfur is the key ingredient to substances which make up human skin collagen, and keratin. While not all antioxidart are created equal, monngaoleifera leaves contain over 30 antioxidants which are well suited for skin health. There are 23 amino acids of which eight are considered essential These are the basis for the construction of new muscle. Scientists have identified Pterygospermin as the active compound in moringa that causes its antibacterial action. Moringa acts as a great natural sleeping aid because it contains the unique natural compound known as Nebedaye Moringa leaves possess anti-tumor and anti-cancel activities, due in part to a compound called niaziminin anti-viral activity in cases of Herpes simplex 1. The glucose-modifying, anti-diabetic effects of moringa may prove of great use amidst a virtual epidemic of Type 2 diabetes and obesity.

BARLEY has a good source of molybdenum, manganese, dietary fiber, selenium, copper, vitamin B1, chromium, phosphorus, magnesium, and niacin thataids in lowering blood cholesterol levels (possibly with propionic acid obtained from the insoluble fiber of barley), protects the intestine and colon (vide its “friendly bacteria” in the large intestine), besides, having anti-cancer, anti-diabetic properties. The propionic acid produced from barley's insoluble fiber may also be partly responsible for the cholesterol-lowering properties of fiber as well as the regularity and intestinal protection. Barley and other whole grains are rich sources of magnesium, a mineral that acts as a co-factor for more than 300 enzymes, including enzymes involved in the body's use of glucose and insulin secretion. The fiber can also help to prevent blood sugar levels from rising too high in people with diabetes.

CHAGA MUSHROOM offer a complex balance of active compounds, delivery mineral structures, and co-agents, more effective to sustaining a healthy immune balance than synthesized isolated compounds that ultimately slows the aging process. It has the highest ORAC (Oxygen Radical Absorbance Capacity) score for natural foods or supplements that protect every cell in the entire body from free-radical damage. It also promote cellular respiration and proliferation, helps regenerate cells and regenerate damaged tissue, promotes the growth of healthy cells and assist in cellular repair, enhances the immune system, maintains optimum alkalinity and pH levels, oxygenates the blood and helps stabilize blood sugar, protects DNA and anti-inflammatory benefits, improve neurological function, digestion and circulation of blood all over the body, alleviates stress and anxiety while increases energy and strength, fights chronic fatigue, reduces muscle and joint pain, supports normal kidney and vision health, balances hormones, manages weight and helps gain muscles, improves lymphocyte count, suppresses allergies, improves memory and concentration, helps optimize cardiovascular health, inhibits lipid peroxidation maintains healthy blood pressure and cholesterol levels, impedes tumor malignancy and boosts natural cancer-fighting ability, helps detoxification of all cells, skin, blood, liver, intestines, colon, kidney, bladder, lymphatic system, lung & respiratory, yeast & fungal, chemicals and heavy metals (including lead and mercury).

BROWN RICE: Brown rice is a rich source of manganese which helps in the synthesis of fatty acids and produces energy from protein and carbohydrates which are helpful in keeping the nervous system strong. Further, manganese is a critical component of superoxide dismutase which is responsible for providing protection against damage from free radicals produced during energy production and balancing the action of calcium in the blood vessels, thereby keeping the blood vessels relaxed and dilated, thus keeping pressure normal and preventing heart attack. It is also a rich source of selenium which helps in thyroid hormone metabolism, and strengthens the immune system. Selenium helps in inducing DNA repair, damaged cell synthesis and inhibits proliferation of cancer cells as well as being an important cofactor of glutathione peroxidase, an antioxidant enzyme, detoxifies the liver of harmful molecules and helps in preventing damage to the cellular DNA and development of cancer cells. Lowers LDL cholesterol and Lower Type-2 Diabetes. Magnesium, another nutrient for which brown rice is a good source, has been shown in studies to be helpful for reducing the severity of asthma, lowering high blood pressure, reducing the frequency of migraine headaches, and reducing the risk of heart attack and stroke.

QUINOA: Quinoa has significantly greater amounts of both lysine and isoleucine (especially lysine), which allows the protein in quinoa to serve as a complete protein source. Besides, it is also rich in RDA nutrients like folate, zinc, and phosphorus. The phytonutrient present in Quinoa provides significant amounts of antioxidants like ferulic, coumaric, hydroxybenzoic, and vanillic acid. Ouinoa also has anti-inflammatory properties due to the presence of phenolic acids like hydroxycinnamic and hydroxybenzoic acids, polysaccharides like arabinans and rhamnogalacturonans and vitamin E like gamma-tocopherol. The antioxidant and anti-inflammatory phytonutrients in quinoa also make it a likely candidate for cancer risk reduction in humans.

OATS contains a specific type of fiber known as beta-glucan which lowers the high cholesterol levels which significantly reduces the risk of cardiovascular disease and stroke. Due to the high fiber content in oats, it reduces the cholesterol levels. The presence of antioxidant compound avenanthramides prevents free radicals from damaging LDL cholesterol, thus reducing the risk of cardiovascular disease. Avenanthramides suppresses production of ICAM-1 (intracellular adhesion molecule-1) and VCAM-1 (vascular adhesion molecule-1), E-selectin, and the secretion of pro-inflammatory cytokines KL-6, chemokines IL-8 and protein MCP-1 (monocyte chemoattractant protein).

SOYBEAN is a source of pepetides like defensins, glycinins, conglycinins and lunasin which helps in improving blood pressure regulation, controlling blood sugar levels, and improving immune function. It is a good source of geistein, an isoflavone which reduces the risk of cancer. It is also rich in phytonutrrients like caffeic, coumaric, ferulic, and sinapic acid. Soybeans are also an important source of the minerals copper, manganese, molybdenum, phosphorus, and potassium; the B vitamin, riboflavin; and omega-3 fatty acids (in the form of alpha-linolenic acid). The area of bone health benefits from soy has been the finding in many studies of improved markers of bone health following consumption of soy.

FLAXSEEDS is a high source of omega-3 fatty acid especially alpha-linolenic acid, or ALA which helps in preventing excessive inflammation of blood vessels, thereby benefitting the cardiovascular system. Further, presence of ligans in flaxseeds imparts antioxidant properties to it. The mucilage content especially the presence of arabinoxylans and galactoxylansin flaxseeds benefits the digestive tract. Decrease the ratio of LDL-to-HDL cholesterol and to increase the level of apolipoprotein A1, and help reduce blood pressure. The antioxidant and anti-inflammatory benefits of flaxseeds aids on cancer prevention.

SUNFLOWER SEEDS: are an excellent source of vitamin E, which is a fat soluble antioxidant and neutralizes free radicals which are harmful to the cell membrane and brain cells. Being an antioxidant, vitamin E helps in preventing cardiovascular diseases, apart from preventing or reducing the occurrence of asthma, osteoarthritis and rheumatoid arthritis vide its anti inflammatory properties.

Sunflower seeds are also rich in manganese which helps in reducing the severity of asthma, lowering high blood pressure, preventing migraine headaches, and reducing the risk of heart attack and stroke. Magnesium is also necessary for healthy bones and energy production. A trace element selenium present in Sunflower seeds induces DNA repair and synthesis in damaged cells, inhibits proliferation of cancer cells and induces their apoptosis, thereby protecting the cells from becoming cancerous. Furthermore, selenium is instrumental in developing body's most powerful antioxidant enzymes, glutathione peroxidase that is used in the liver to detoxify a wide range of potentially harmful molecules.

WALNUTS are high sources of anti-inflammatory nutrients like omega-3 fatty acids including alpha-linolenic acid, phyto nutrients like tannins, phenolic acids, and flavonoids and quinines like juglone. The presence of anti-inflammatory nutrients (alpha-linolenic acid) helps the cardiovascular system and regulates the blood composition as well as blood pressure. It reduces problems in metabolic syndrome and is beneficial in the treatment of type-2 diabetes. The antioxidant properties of walnuts help lower risk of chronic oxidative stress. Also, walnut helps to keep bones strong and prevents prostrate and breast cancer.

BLACK BEANS are also rich sources of phytonutrients and anthocyanin flavonoids like delphinidin, petunidin, malvidin, kaempferol and quercetin. These phytonutrients and flavonoids impart both antioxidant and anti-inflammatory properties to the black beans. Black beans are also sources of hydroxycinnamic acids ferulic, sinapic, and chlorogenic acid, as well as numerous triterpenoids. Antioxidant minerals like zinc and manganese are also plentiful in black beans. Also, found in black beans is omega-3 fatty acids that helps control blood pressure and cardio-vascular system. Black beans are rich both in proteins as well as fibres. This combination is beneficial for smooth functioning of the digestive tract, in regulating the blood sugar system and the cardiovascular system.

COCONUTS have anti-viral, anti-bacterial, anti-fungal, and anti-parasite properties. It improves digestion and absorption of nutrients, vitamins, and minerals in the body thereby providing a natural source of quick energy in the body. It stimulates insulin secretion thereby guards against diabetes. It protects the body from degeneration by removing the free radicals present in the system.

GOJI BERRIES are excellent source of antioxidants such as polyphenols, flavonoids, carotenoids, vitamins like A, C and E apart from having potassium, zinc, iron, copper and riboflavin in them. They are also rich in polysaccharides which are a primary source of dietary fiber. Gogi berries, due to the presence of antioxidants, help neutralize the cell-damaging effects of free radicals and help guard us from degenerative diseases such as rheumatoid arthritis, Alzheimer's disease and most types of cancer. It also contain 8 polysaccharides, a primary source of dietary fiber.

HONEY: Honey is a real food that has been accessible to humans throughout evolutionary history and can still be obtained in its natural form. Existence of fructose and Omega-6 fatty acids despite being linked to health issues when isolated, in “real foods” have a completely different effect. Honey raises blood sugar less than dextrose (glucose) and sucrose (glucose and fructose). It reduces C-Reactive Protein (CRP)—a marker of inflammation, lowers LDL cholesterol, blood triglycerides and raises HDL cholesterol and also lowers Homocysteine, another blood marker associated with disease. Honey also contains an abundance of various antioxidants that are associated with improved health and lower risk of disease. Two human studies revealed that consumption of buckwheat honey increases the antioxidant value of the blood

BLACK SEEDS Black cumin used as a spice is considered a natural remedy for asthma, hypertension, diabetes, inflammation, cough, bronchitis, headache, eczema, fever, dizziness, and influenza. The seeds are known to be carminative, stimulant, and diuretic. Similarly, seeds of herbaceous plant are used in the prevention of inflammation, antioxidant activities, antimicrobial activity, and anti -carcinogenic and antiulcer activity.

U.S. PATENT DOCUMENTS

| | | |
|---|---|---|
| 6,482,449 | Nov. 19, 2002 | Brown |
| 6,849,281 | Feb. 1, 2005 | Bodor |
| 6,902,739 | Jun. 7, 2005 | McPeak |
| 7,166,313 | Jan. 23, 2007 | Diddle |
| 7,592,028 | Sep. 22, 2009 | Vodovotz |
| 7,803,844 | Sep. 28, 2010 | Lee |
| 7,977,319 | Jul. 12, 2011 | Levine |
| 7,993,686 | Aug. 9, 2011 | Bird |
| 8,017,172 | Sep. 13, 2011 | Arndt |
| 8,470,352 | Jun. 25, 2013 | Liu |
| 8,613,971 | Dec. 24, 2013 | Finocchiaro |
| 8,623,841 | Jan. 7, 2014 | Addis |
| 8,628,816 | Jan. 14, 2014 | Henry |
| 8,652,544 | Feb. 18, 2015 | Minatelli |
| 8,747,935 | Jun. 10, 2014 | Villota |
| 8,877,267 | Nov. 4, 2014 | Astrup |
| 8,802,161 | Aug. 12, 2014 | Mazzio |
| 8,852,651 | Oct. 7, 2014 | Al-Shehry |
| 8,980,949 | Mar. 17, 2015 | Bar-Yosef |
| 8,524,245 | Sep. 3, 2013 | Yu |
| 8,962,689 | Feb. 24, 2015 | Mingrone |
| 8,937,049 | Jan. 20, 2015 | Stellinggwerff |
| 8,932,651 | Jan. 13, 2015 | Lax |
| 8,846,126 | Sep. 30, 2014 | Baseeth |
| 8,524,249 | Sep. 3, 2013 | Wegkamp |

### OTHER REFERENCES

Food As Medicine, How Are Food and the Environment Related?, How Does Food Impact Health? What Should I Eat for My Specific Condition? http://www.takingcharge.csh.umn.edu/our-experts/karen-lawson-md

### DRAWINGS

Non Applicable

### SEQUENCE LISTING

Non Applicable

While there have been shown and described what are at present, the preferred embodiments of the invention, it will be obvious to those skilled in the art that various changes and modifications may be made therein without departing from the scope of the invention as defined by the appended claims.

For a better understanding of the present invention, together with other and further objects, advantages and capabilities thereof, reference is made to the following disclosure and appended claims.

I claim:

1. A natural/organic food or nutraceutical composition comprising of:
barley, brown rice, oats, quinoa, whole wheat, soybeans, black beans, honey, flaxseed, sunflower seeds, walnuts, black seed, moringa, goji berries, coconut, seaweeds and chaga mushrooms;
wherein the said ingredients are combined in various proportions and in different combinations and prepared in various forms of food products for providing daily nutritious balanced diet with critical components like proteins, enzymes, minerals, vitamins, fiber, fatty acids and iodine to bring and maintain general well-being of the human body.

2. A natural food or nutraceutical composition according to claim 1, wherein the mushroom selected is Chaga mushroom.

3. A natural food or nutraceutical composition according to claim 1, wherein one of the various forms of food product is pasta and its derivatives like ravioli, cannelloni, lasagna, long and short pasta.

4. A natural food or nutraceutical composition according to claim 1, wherein one of the various forms of food product is bread and its derivatives like pizza, tortillas, crackers, arepas and empanadas.

5. A natural food or nutraceutical composition according to claim 1, wherein one of the various forms of food product is cereals.

6. A natural food or nutraceutical composition according to claim 1, wherein the ingredients are mixed in the following combination and proportion by weight for the preparation of dough for pasta:

| Ingredient | Percentage by weight (gram) |
|---|---|
| Seaweeds | 10 |
| Black seeds | 24 |
| Sunflower | 100 |
| Flaxseeds | 50 |
| Soyabean | 170 |
| Oats | 120 |
| Barley | 120 |
| Brown Rice | 400 |
| Total | 1000 |

7. A natural food or nutraceutical composition according to claim 1, wherein the ingredients are mixed in the following combination and proportion by weight preparation of dough for pasta:

| Ingredient | Percentage by weight (gram) |
|---|---|
| Chaga Mushroom | 50 |
| Seaweeds | 10 |
| Black seeds | 35 |
| Flax seeds | 100 |
| Moringa | 100 |
| Sunflower seeds | 100 |
| Brown rice | 600 |
| Walnuts | 5 |
| Total | 1000 |

8. A natural food or nutraceutical composition according to claim 1, wherein the ingredients are mixed in the following combination and proportion by weight preparation of dough for pasta:

| Ingredient | Percentage by weight (gram) |
|---|---|
| Seaweed | 5 |
| Black seeds | 10 |
| Flaxseeds | 100 |
| Echinacea | 15 |
| Oats | 150 |
| Barley | 150 |
| Quinoa | 150 |
| Brown rice | 420 |
| Total | 1000 |

9. A natural food or nutraceutical composition according to claim 1, wherein the ingredients are mixed in the following combination and proportion by weight preparation of dough for pasta:

| Ingredient | Percentage by weight (gram) |
|---|---|
| Brown rice | 520 |
| Black beans | 300 |
| flaxseeds | 100 |
| chaga | 50 |
| Black seeds | 25 |
| Seaweeds | 5 |
| Total | 1000 |

10. A method of making a food or nutraceutical composition according to claim 6, 7, 8 or 9 comprising the steps of preparing the dough for pasta involving pulverizing the said ingredients and placing & mixing them in a conventional mixing device thereby, slowly adding 400 gram water and adding 50 gram of crushed flaxseeds. After waiting for 5 minutes, the next step involves placing the well-blended mixture in the pasta machine for 10 minutes after which the pasta sheets of different lengths and thicknesses gets prepared, whereby the prepared pasta sheets are placed in different machines accordingly, to get the pastas of the desired forms like ravioli, cannelloni, lasagna, long and short pastas. The pasta is then dried for 12 hours.

11. A method of making a food or nutraceutical composition according to claim 10, wherein, before consumption of the said dried pasta, it is steamed at low boiling point for 5-6 minutes.

12. A natural food or nutraceutical composition according to claim 1, wherein the ingredients are mixed in the following combination and proportioned by weight for the preparation of dough for bread/pizza/tacos/tortillas/crackers/arepas/empanadas:

| Ingredient | Percentage by weight (gram) |
|---|---|
| Seaweeds | 2 |
| Blackseeds | 3 |
| Sunflower Oil | 15 |
| Flaxseeds | 10 |
| Soybean | 20 |
| Brown Rice | 100 |
| Total | 152 |

13. A natural food or nutraceutical composition according to claim 1, wherein the ingredients are mixed in the following combination and proportion by weight for the preparation of dough for bread pizza/tacos/tortillas/crackers/arepas/empanadas:

| Ingredient | Percentage by weight (gram) |
|---|---|
| Chaga Mushrooms | 8 |
| Seaweeds | 2 |
| Black seed | 5 |
| flaxseeds | 15 |
| Moringa | 25 |
| Sunflower oil | 13 |
| Walnuts | 2 |
| Barley | 80 |
| Total | 150 |

14. A natural food or nutraceutical composition according to claim 1, wherein the ingredients are mixed in the following combination and proportion by weight for the preparation of dough for bread pizza/tacos/tortillas/crackers/arepas/empanadas:

| Ingredient | Percentage by weight (gram) |
|---|---|
| Black seeds | 5 |
| Flaxseeds | 5 |
| Echinacea | 10 |
| Oats | 10 |
| Barley | 80 |
| Quinoa | 30 |
| Sunflower oil | 10 |
| Total | 150 |

15. A method of making a food or nutraceutical composition according to claim 12, 13 or 14 comprising the steps of preparing the dough for bread/pizza/tacos/tortillas/crackers/arepas/empanadas involving pulverizing the said ingredients and placing & mixing them in a conventional mixing device thereby, slowly adding 90 gram water and adding 20 gram of sunflower oil,
wherein, the next step involves placing the well-blended mixture in the mixing device for 10 minutes, which produces the required dough for making different products, wherein filling those different products shapes with nutritionally balanced food fillings, thereby producing the final nutraceutical composition for the bread/pizza/tacos/tortillas/crackers/arepas/empanadas.

16. A method of making a food or nutraceutical composition according to claim 15, wherein the raw food product, cracker, is steam baked for 1-2 minutes and sold as such, whereas the raw food products of bread/pizza/ tacos/tortillas/arepas/empanadas, as herein produced, are frozen and sold, and during consumption are steam baked for 15 minutes at a temperature of 200 degrees Fahrenheit for pizza and bread and 1-2 minutes for tacos, tortillas, arepas and empanadas.

17. A natural food or nutraceutical composition according to claim 1, wherein the ingredients are mixed in the following combination and proportioned by weight for the preparation for cold /hot cereals and snack bars.

| Ingredient | Percentage by weight (gram) |
|---|---|
| Oats | 600 |
| Barley | 80 |
| Soybean | 80 |
| Brown rice | 50 |
| Flaxseeds | 50 |
| Honey | 50 |
| Sunflower Oil | 50 |
| Blackseeds | 20 |
| Seaweeds | 10 |
| Gogi berries | 10 |
| Total | 1000 |

18. A natural food or nutraceutical composition according to claim 1, wherein the ingredients are mixed in the following combination and proportioned by weight for the preparation for hot/cold cereals and snack bars:

| Ingredient | Percentage by weight (gram) |
|---|---|
| Oats | 600 |
| barley | 90 |
| Brown Rice | 50 |
| Moringa | 50 |
| Chaga Mushrooms | 50 |
| Flax seeds (crushed) | 50 |
| Sunflower Seeds | 50 |
| Gogi Berries | 35 |
| Sea Weeds | 10 |
| Walnuts | 10 |
| Black seeds | 5 |
| Total | 1000 |

19. A natural food or nutraceutical composition according to claim 1, wherein the ingredients are mixed in the following combination and proportion by weight for hot/cold cereals and snack bars:

| Ingredient | Percentage by weight (gram) |
|---|---|
| Oats | 600 |
| Quiona | 100 |
| Barley | 90 |
| Brown Rice | 80 |
| Honey | 50 |
| Flaxseeds (crushed) | 50 |
| Black seeds | 15 |
| Seaweeds | 5 |
| Coconut | 10 |
| Total | 1000 |

20. A method of making a food or nutraceutical composition according to claim 17, 18 or 19, comprising the steps of preparing the granola for cold cereals and snack bar involving pulverizing the above ingredients and mixing and placing them on a tray of the conveyer belt and wet steam baking the mixture at 150 degree Fahrenheit for 1 minute, therein, adding 50 gram of honey and dry steam baking for another minute.

21. A method of making food or nutraceutical compositions, according to claim 10,11,15,16 or 20 wherein, the methods are mechanical processes designed as alternative solutions to chemically processed food, whereby, the food retain the nutritional value creating a “disease preventive food” for “dietary interventions.”

22. A method of making food or nutraceutical composition, according to claim 10, 11,15,16, 20 or 21 wherein the said methods are mechanical processes of steam cooking which protects the food from losing its nutrients and medicinal properties, whereby creating a “culinary intervention”, and another “disease prevention strategy”.
